(19) United States
(12) Patent Application Publication
(10) Pub. No.: US 2009/0285780 A1
(43) Pub. Date: Nov. 19, 2009

(54) PEG LINKER COMPOUNDS AND BIOLOGICALLY ACTIVE CONJUGATES THEREOF

(76) Inventor: Chyi Lee, Princeton Junction, NJ (US)

Correspondence Address: FOX ROTHSCHILD LLP;PRINCETON PIKE CORPORATE CENTER, 2000 Market Street, Tenth Floor, Philadelphia, PA, 19103, US

(21) Appl. No.: 12/302,238

(22) PCT Filed: May 24, 2007
(86) PCT No.: PCT/US07/69697

§ 371 Date: Nov. 24, 2008

Related U.S. Application Data
(60) Provisional application No. 60/808,175, filed on May 24, 2006.

(51) Int. Cl. A61K 47/48; C07C 237/12; C07C 237/06; C07C 231/00; A61K 38/21; A61K 38/00

(52) U.S. Cl. 424/85.4; 562/561; 564/153; 514/2; 514/772

(57) ABSTRACT

PEG linker compounds and biologically active conjugates thereof having mixed functional group linkages attached to at least one PEG moeity, and a coupling group for attaching a biologically active molecule. The PEG mixed linkages can be the combinations of stable, or labile, or releasable, or stable and labile, or stable and releasable, or releasable and labile covalent linkages. The mixed functional linkages of the PEG linker compounds consist of different organic functional groups, which have non-equivalent half-life in plasma and, hence, they have different release rates in blood. The present invention also provides for attachment of novel PEG linker compounds with mixed functional linkages for Pegylation of biologically active molecules to produce Probiomolecule-PEG constructs. The Probiomolecule-PEG construct is the prodrug of biomolecule-PEG conjugate or biomolecule. The Probiomolecule-PEG conjugate will gradually lose portions (or all) of its PEG polymers in vivo to convert into smaller size biomolecule-PEG conjugate (or biologically active molecule), thereby increasing their biological activity in vivo.

This application claims the benefit of U.S. patent application 60/808,175 filed May 24, 2006, which is herein incorporated by reference in its entirety.

### FIELD OF THE INVENTION

The present invention generally relates to PEG linker compounds containing mixed functional group linkages and biologically active conjugates thereof.

### BACKGROUND OF THE INVENTION

Coupling of polyethylene glycol (PEG) to biologically active molecules termed “Pegylation” are used in the delivery biologically active molecules usually proteins and small molecules by modifying the pharmacokinetic (PK) and pharmacodynamic (PD) properties of the biologically active molecules. Pegylation increases the size and molecular weight of proteins and small molecules resulting in the extension of their half-life in plasma. In general, Pegylation may alter the physicochemical properties of the proteins and therapeutic molecules resulting in decreased bioactivity of the parent proteins and organic therapeutic molecules. It is therefore desirable to optimize the PK and PD properties of PEG-protein conjugates for achieving their maximum therapeutic efficacies.

Covalent attachment of PEG to biologically active molecules in the prior art, including linear and branched PEG polymers, has been achieved in a vast majority of cases utilizing amino groups of biologically active molecules as sites of attachment. US Patent Application 20030190304 to Thompson et al. describes Peglation reagents. U.S. Pat. No. 7,030,278 to Harris et al. describes certain PEG derivatives with proximal reactive groups. Certain non-antigenic branched polymer conjugates are described in U.S. Pat. No. 5,643,575 to Martinez et al. Certain multi-armed PEG polymers are described in the US Patent Application 20050033058 to Huang et al. Certain active carbonates for modification of polypetides are disclosed in U.S. Pat. No. 5,122,614 to Zalipsky.

Attachment involving in the conversion of the amino group usually result in the loss of positive amino charges of the biologically active molecules. Such structural changes of the pegylated biologically active molecules may change PK and PD and decrease their bioactivity. Another issue prevalent in pegylated biologically active conjugates is their aggregation and precipitation which often limit the application of Pegylation.

Given the above, it is desirable to have pegylated biologically active conjugates that overcome these deficiencies.

### SUMMARY OF THE INVENTION

The present invention fills the foregoing need by providing PEG linker compounds with mixed functional linkages and new methodologies for the Pegylation of biologically active molecules to maximize their bioactivities and concentrations in plasma.

One aspect of the present invention provides for a PEG linker compounds comprising mixed functional group linkages attached to at least one PEG moeity, and a coupling group capable of reacting with a biologically active molecule.

In an embodiment the mixed functional linkages are the combination of various covalent bonds including, but are not limited to, carboxylic ester, carbonate, carbamate (urethane), carbamide (urea), amide, sulfide, and disulfide. In other embodiments of the invention, the PEG linker compounds may contain mixed functional linkages for the conjugation of biologically active molecules.

Another aspect of the present invention provides for a pyramidal PEG linker compound comprising the formula:

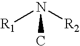

wherein R₁, R₂ represents a functional group that is attached to a PEG moiety selected from the group consisting of PEG polymers, PEG derivatives and PEG lipids; and

C is a coupling group, which is capable of reacting with a biologically active molecule.

In an embodiment the coupling group is capable of reacting with the amino, thio, carboxyl or aldehyde groups of the biologically active molecule. In another embodiment, the pyramidal PEG linker compound may contain branched PEG polymers for the conjugation of biologically active molecules.

Another aspect of the present invention provides for a PEG linker compound comprising the formula:

Pn-Ln-R—C

wherein P is a PEG moiety selected from the group consisting of PEG polymer, PEG derivative, and PEG-lipid;

L is functional linkage moiety;

n is the number of different kinds of linkage moieties and n≧2;

R is a compound base structure bonded to at least two different kinds of functional groups for linking to P; and

C is a coupling group capable of reacting with a biologically active molecule.

Another aspect of the invention provides for a charged PEG linker compound comprising functional groups having a positive charge and/or a negative charge.

Another aspect of the invention provides for a method of preparing a biologically active conjugates comprising:

reacting a biologically active molecule with a PEG linker compound comprising mixed functional group linkages attached to at least one PEG moeity, and a coupling group for reacting with the biologically active molecule.

Another aspect provides for the methods of forming above PEG linker compounds.

Further aspects of the present invention provides for biologically active conjugates formed by the reaction of the coupling groups of the PEG linker compounds with biologically active molecules.

Another aspect provides for compositions comprising the PEG linker compounds and biological molecules.

### DETAILED DESCRIPTION OF THE INVENTION

PEG linker compounds of the present invention also contains an active functional group that is capable of attaching to different functional sites of biologically active molecules. The PEG linker compounds containing mixed functional linkages have two or more different covalent linkages. The PEG mixed linkages can be the combinations of stable, or labile, or releasable, or stable and labile, or stable and releasable, or releasable and labile covalent linkages. The mixed functional linkages of the PEG linker compounds consist of different organic functional groups, which have non-equivalent half-life in plasma and, hence, they have different release rates in blood. Therefore, optimization of the PK and PD properties of the Pegylated biologically active molecules can be achieved by conjugating biologically active molecules with various PEG mixed functional linkages. One of the advantages of using the PEG mixed linkages is that the different kinds of chemical functional groups of the PEG mixed linkages have variable half-life in blood plasma. This property of the mixed linkage PEG linker compounds provides new methodologies to increase the effectiveness in PK and PD of the Pegylated molecules. The resulting increased effectiveness in PK and PD properties may result in increasing efficacy and safety for the biologically active molecules. The biologically active molecules include and are not limited to proteins, bio-polymers, amino acids and organic therapeutic molecules.

Attachment of coupling groups of the PEG linker compounds to biologically active molecules containing functional groups such as amino groups of the proteins results in the conversion of the amino groups and resulting in the loss of the positive charge on the amino groups. In order to overcome the loss of charge in biologically active molecules the PEG linker compounds of the present invention may incorporate a tertiary amine into the compound structure that is attached to the PEG moieties. The PEG moieties referred to in this application includes PEG polymers, derivatives and PEG lipids. The PEG polymers can be linear, branched or multi-armed.

In an embodiment the pyramidal shape of the tertiary amine with groups that are connected to the PEG moieties can retain the charges of the biologically active molecules after attaching the coupling group to the amino groups of the biologically active molecules. Also, the pyramidal shape of the tertiary amine provide for a center of rotation for the groups that are attached to PEG moieties. It is believed that the low potential energy of N—C bond provides for the freedom in rotation and the attached PEG moieties may enhance the shielding effects of the PEG groups to provide more reduction in proteolysis and immunogenicity to biologically active molecules. Pyramidal multi-branched PEG linker compounds containing tertiary amines and multiple PEG strands which can provide charges and better shielding effects to the biologically active molecules they are coupled with. This type of PEG linker compounds is useful for the pH sensitive biologically active molecules and can potentially extend the half-life of PEG-biologically active conjugates in plasma in comparison with the biologically active molecules attached with conventional PEG polymers.

The pyramidal PEG linker compounds of the present invention contain an active functional group, which is capable of attaching to the biologically active molecules, including proteins, peptides, antibody, antibody fragment, single chain antibody, hormones, enzymes, oligonucleotides, saccharine, lipids, biomaterials, liposomes and particulates, affinity ligands and cofactors, and small molecule drugs and agents. The pyramidal multi-branched PEG linker compounds of the present invention containing multiple tertiary amines carry multiple positive charges and PEG strands may provide new methodologies for the process of Pegylation for the pH sensitive proteins to reduce the aggregation. For example, it has been reported that Interferon β-1b is susceptible to aggregation. The pyramidal multi-branched PEG may provide solutions for the process of Pegylation of Interferon β-1b, and improve the stability of the PEG-Interferon-β-1b conjugate. In addition, the multiple branched PEG strands can form a better protective shell around the biologically active molecule that shield the biologically active molecule from immunogenic recognition and reduce the degradation by proteolytic enzymes. Pyramidal PEG linker compounds containing tertiary amines carry positive charges and single or multiple permanent PEG linkages. Attachment of pyramidal PEG linker compounds to amino groups of biologically active molecules does not change the charges of the biologically active molecules.

Aggregation and precipitation for PEG-biologically active conjugates often limit the applications of Pegylation. The charged PEG branched linker compounds may contain multiple PEG polymer strands and multiple charges. PEG linker compounds of this type provide several advantages for Pegylation of the biologically active molecules which may have problems relating to aggregation, solubility, immunogenicity or proteolysis. This type of charged PEG linker compounds include linear, branch, multi-arm, and star shape PEG polymers. The positive or negative charged functional groups are usually implanted on the linker compound moiety of the PEG-derivatives. The negative charged functional groups include carboxylic, sulfonic, or phosphoric acids, or the functional groups capable of offering negative charges after reacting with amino groups of the biologically active molecules, such as carboxylic anhydride, sulfonic anhydride, or phosphoric anhydride. The positive charged functional groups on the PEG-linker compounds can be tertiary amines, quarternary amines, or heterocyclic amines. The charged PEG polymers contain an active functional group that is capable of attaching to the biological molecules.

In addition, the charged PEG linker compounds of the present invention carrying positive or negative charged functional groups may prevent aggregation and precipitation as well. The positive or negative charged functional groups are usually present in the PEG-linker compound portion and are synthesized by the reaction of different functional groups. A person skilled in the art will recognize that the negative charged functional groups can be carboxylic, sulfonic, phosphoric acids, or the functional groups capable of offering negative charges after reacting with amino groups of the biologically active molecules, such as carboxylic anhydride, sulfonic anhydride, or phosphoric anhydride. The positive charged functional groups on the PEG-linker compounds can be tertiary amine, quarternary amine, or heterocyclic amines. The charged PEG linker compounds may improve the process of Pegylation for certain biologically active molecules and also may increase the stability of the Pegylated proteins in storage. Additionally, the charged branched linker compounds contain multiple PEG polymer strands that enhance the shielding effects on Pegylated molecules to provide the additional reduction of proteolysis and immunogenicity.

In another embodiment of the invention, the PEG polymers may carry multiple charges for the conjugation of biologically active molecules.

The molecular weights of polyethylene glycol (PEG) or methoxy polyethylene glycol (mPEG) in this invention are in the range of 200 to 150,000 daltons for PEG linker compounds with mixed functional linkages, pyramidal PEG linkers, pyramidal multi-branched PEG linkers, or charged PEG linkers.

For PEG linker compounds with mixed functional linkages, pyramidal PEG linkers, pyramidal multi-branched PEG linkers, or charged PEG linker compounds in this invention, each PEG linker compound contains an active functional group that can be various electrophilic or nucleophilic functional groups, which can be attached to the bio-molecules, including proteins and small molecules.

The amino-reactive electrophilic groups, can be N-hydroxysuccinimide (NHS) ester, p-nitophenyl ester, succinimidyl carbonate, p-nitrophenyl carbonate, succinimidyl urethane, isocyanate, isothiocyanate, acyl azide, sulfonyl chloride, aldehyde, carbonate, imidioester or anhydride. The thio-reactive groups are maleimide, haloacetyl, alkyl halide derivatives, aziridine, acryloyl derivatives arylating agents or thio-disulfide exchange reagents.

The nucleophilic functional groups, including amine, hydrazide, carbazate, acyl hydrazide, semicarbamate or hydrazine, can undergo reactions with aldehyde or carboxyl groups on proteins.

Biologically active molecules of interest which can be attached with the PEG linker compounds with mixed linkages, pyramidal PEG linkers, pyramidal multi-branched PEG linkers, or charged PEG linker compounds in this invention include, but not limited to, proteins, peptides, oligonucleotides, saccharides, lipids, lipsomes and particulates, biomaterials, pharmaceuticals, vitamins, nucleic aids, amino acids, polypeptides, enzyme cofactors, steroids, carbohydrates, heparin, metal containing agents, receptor antagonists, receptor agonists, receptors or portions of receptors, extracellular matrix proteins, cell surface molecules, antigens, haptens, and small molecules. Proteins of interest include, without limitation, cytokines, enzymes, growth factors, monoclonal antibody, antibody fragments, single-chain antibody, albumin, immunoglobulins, clotting factors, somatropin, amylase, lipase, protease, cellulose, urokinase, galactosidase, staphylokinase, hyaluronidase, tissue plasminogen activator and others.

The present invention also provides biologically active conjugates containing a biologically active molecule and at least one of PEG linker compounds with mixed linkages, pyramidal PEG linkers, pyramidal multi-branched PEG linkers, or charged PEG linker compounds as disclosed herein. The biologically active conjugates of the present invention may be synthesized by reacting the biologically-active molecules with the PEG linker compounds with mixed linkages, pyramidal PEG linkers, pyramidal multi-branched PEG linkers, or charged PEG linker compounds as disclosed herein in a manner known in the art.

Potential biologically active molecules for the applications of PEG mixed linkages include, but not limited to, epoetin alfa, filgrastim, etanercept, interferon α-2a, interferon α-2b, interferon alfacon-1, interferon β-1a, interferon β-1b, interferon γ-1b, interleukins, insulin, urokinase, streptokinase, uricase, superoxide dismutase, asparaginase, arginine deaminase, glucocerebrosidase, galacosidase, retelapse, rasburicase, laronidase, oprelvekin, dornase α, collagenase, anistreplase, agalsidase, growth factors, hemoglobin, blood factors VII, VIII, and IX, etc.

I. PEG Linker Compounds with Mixed Functional Linkages

The mixed functional linkages of the PEG linker compounds are the combinations of different functional groups, such as carboxylic ester, carbonate, carbamate (urethane), carbamide (urea), amide, imide, sulfide, disulfide, sulfonic ester, phosphoric ester, or the pH or enzyme dependant releasing linkages.

In general, the compounds used for the synthesis of PEG mixed functional linker compounds contain three or more different functional groups, and they can be the combinations of amino, hydroxyl, thio, carboxyl, phosphoryl, sulfonyl groups, or the same functional groups having protective groups attached.

Examples like the amino acids, including serine, threonine, cysteine, tyrosine, histidine, and arginine can undergo nucleophilic substitution reactions with various PEG electrophilic derivatives to yield the desired PEG mixed functional linkages. These amino acids or chemical species contain various nucleophiles, such as hydroxy, amino, thio, imidazo, and guanidine groups, that can react with PEG electrophilic reagents.

Peptides, preferably dipeptide or tripeptide, containing those amino acids described above can also be used for the synthesis of PEG linker compounds with mixed functional linkages, for example, the dipeptides having the following formula correspond to the invention:

H₂N-Amino Acids-R′

R′: amino acids containing two different functional groups, e.g. serine, threonine, cysteine, tyrosine, histidine, arginine.

Other chemical compounds having three with at least two different kinds of functional groups or multiple various functional groups, can be used for the synthesis of PEG mixed functional linkages, such as Tricine, Hydroxy glutamic acid, 4-Amino-2-hydroxybutyric acid, L-xylo or 5-Amino-3-hydroxy-pentanoic acid ethyl ester. The PEG linker compounds containing mixed functional linkages in this invention correspond to the following formula:

Pn-Ln-R—C

P: PEG, PEG derivatives, PEG-lipid
L: functional linkage
n: number of different kinds of linkages, n≧2
R: compound base structure bonded to at least two different kinds of functional groups for linking PEG
C: coupling group for conjugation

For example, if n=2, the formula is shown as follows:

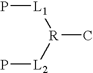

Examples of the synthesis of the PEG mixed linkages:

Hydroxy (OH) groups react with PEG urethane succinimidyl ester to form PEG carbamate (urethane) linkage. And hydroxyl groups react with PEG-p-nitro-phenyl carbonate or PEG-succinimidyl carbonate (SC-PEG) to form PEG-carbonate linkage.

Amino groups, including alkyl amine, aromatic amine, imidazole and guanidine, react with succinimidyl ester of urethane PEG to form carbamide (urea) linkage. And amino groups react with PEG-p-nitrophenyl carbonate or PEG-succinimidyl ester (SC-PEG) to form carbamate (urethane) linkage.

Thio groups (SH) reacts with PEG-maleimide or PEG-iodo acetyl group to form sulfur-maleimide stable linkage.

PEG derivatives containing electrophiles capable of reacting with nucleophiles:

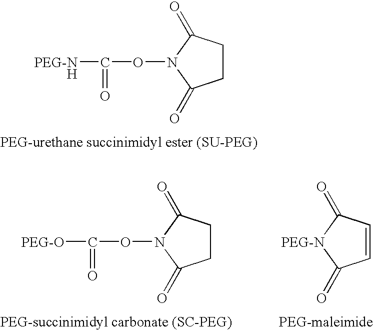

Examples of the covalent functional groups for the PEG mixed linkages:

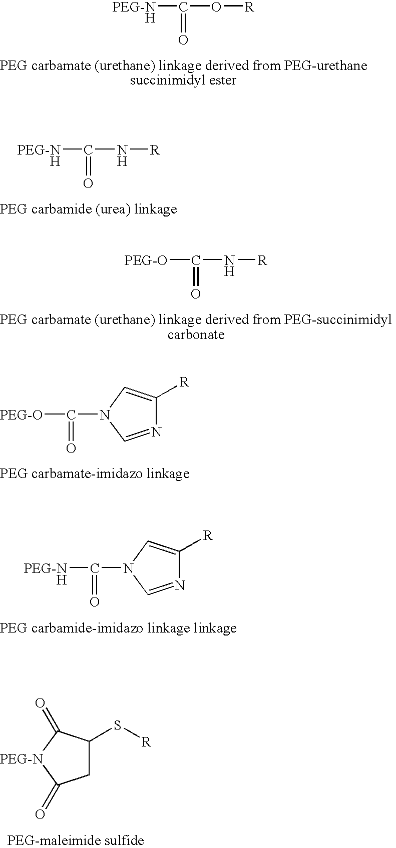

The following three serine-PEG derivatives are used to illustrate the formula of PEG linker compounds with mixed linkages:

One mole of serine reacts with two moles of mPEG-urethane-N-hydroxy succinimide ester to yield PEG-cysteine mixed carbamate and carbamide linkages. The carboxyl group can be activated to an active ester for coupling to the bio-molecules. Other activating groups for carboxyl well known in the art can also be used.

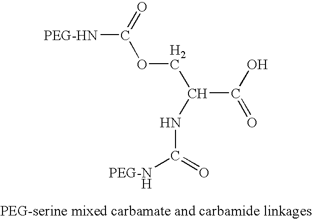

One mole of serine reacts with two moles of mPEG-p-nitrophenyl carbonate or mPEG succinimidyl carbonate to offer PEG-cysteine mixed carbamate and carbonate linkages.

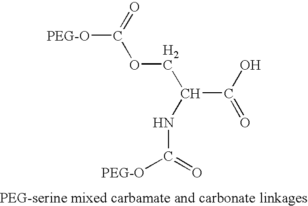

One mole of serine reacts with two moles of succinimidyl ester of carboxymethylated mPEG to generate PEG-cysteine mixed amide and ester linkages.

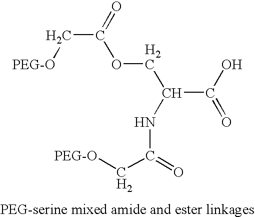

Any amino acid capable of reacting with mPEG could be used instead of serine.

Examples 1, 2 and 3 illustrate certain aspects of the invention with regard to the PEG linker compound with mixed functional linkage, and should not to construed as limiting the scope of the invention.

### II. Pyramidal PEG Linkers

The pyramidal PEG linker compound contains at least one tertiary amine, and the nitrogen containing a pair of electrons is on the top of the triagonal pyramid. The formula is as follows:

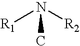

where R₁ and R₂ is the functional group that is attached to the PEG polymers, and they can have equivalent or non-equivalent PEG linkages. If R₁ and R₂ have different kinds of functional linkages, then it is the pyramidal PEG mixed linkages. The mixed functional groups can be a combination of the following covalent bonds: carboxylic ester, carbonate, carbamate (urethane), carbamide (urea), amide, sulfide, and disulfide.

“C” represents the coupling group, which is capable of reacting with the biologically active molecule containing functional groups such as amino, thio, carboxyl or aldehyde groups. The coupling groups can be either electrophiles or nucleophiles.

Compounds include, but not limited to, N-(2-Hydroxyethyl) iminodiacetic acid, bicine, Nα,Nα-Bis(Carboxymethyl)-L-lysine can be used to synthesize the pyramidal PEG linkers.

One skilled in the art would realize that the N group has a lone pair for imparting the positive change by forming quaternary compounds.

Examples 4, 5, and 6 illustrate certain aspect of the invention with regard to the pyramidal PEG linkers, and should not to construed as limiting the scope of the invention.

### III. Pyramidal Multi-Branched PEG Polymers

The advantages of attaching pyramidal multi-branched PEG to proteins over the conventional PEG polymer are that the pyramidal multi-branched PEG can increase the size of the PEG-protein conjugate and enhance shielding effect on protein, and that may result in more decreasing in renal clearance, proteolysis, antigeneicity, and opsonisation, and therefore a favorable drug efficacy and safety can be achieved. In addition, some pyramidal multi-branched PEG polymers containing more than one tertiary amine can carry more charges and thus, it may prevent certain PEG-protein conjugates from aggregating in aqueous solution.

Compounds can be used to synthesize the pyramidal multi-branched PEG polymers include, but not limited to, N-(2-hydroxyethyl)ethylenediaminetriacetic acid, ethylenediaminetetraactic acid triethyl ester, and iminodisuccinic acid.

The active ester of N-(2-hydroxyethyl)ethylenediaminetriacetic mPEG3 can be synthesized using similar procedures as described in examples 4, 5, and 6. This PEG linker compound carries two positive charges with 3 PEG polymer strands attached to two tertiary amines.

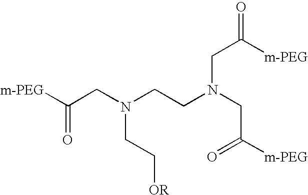

where OR represents the active esters for protein conjugation, such as p-nitrophenyl carbonate, succinimidyl carbonate or imidazole carbamate

### IV. Charged PEG Linkers

Aggregation and precipitation for PEG-protein and PEG-small molecules often limit the applications of Pegylation. The PEG carrying positive or negative charged functional groups can be used for solving the problems of aggregation or solubility for Pegylated bio-molecules. The positive or negative charged functional groups are usually implanted on the linker compound region of the PEG-derivatives. The negative charged functional groups can be carboxylic, sulfonic, phosphoric acids or the functional groups capable of offering negative charges after reacting with amino nucleophiles, such as carboxylic anhydride, sulfonic anhydride, or phosphoric anhydride. And the positive charged functional groups on the PEG-linker compounds can be the tertiary amine or ammonium complex.

The linear or branched PEG containing one or more than one free carboxyl, sulfuric, phosphoric groups after attaching to proteins can result in the more acidic PEG-protein conjugates. Theses types of PEG polymers carries more negative charges and therefore enhance the solubility of the PEG-protein conjugates at higher pH. It is extremely useful for proteins having aggregation problems at higher pH, and attachment of this type of PEG polymer can potentially prevent the aggregation of PEG-proteins.

Many chelating compounds contain multiple tertiary amines can carry multiple positive charges, and their multiple hydroxyl, thio and carboxyl groups can be connected to PEG polymers. These types of chelator-PEG polymers are multi-branched PEG polymers carrying multiple charges. Examples are Diethylenetriamine pentaacetic acid (DTPA), Ethylenediamine tetraacetic acid (EDTA), Triethylenetetramine-N,N,N′,N′,N″,N″-hexaacetic acid (TTHA), 1,3-Diamino-2-hydroxypropane-N,N,N′,N′-tetraacetic acid, and N-(2-Hydroxyethyl)ethylenediamine triacetic acid.

The charged linker compounds may also be used to form nanoparticles of conjugates. The nanoparticles may be comprised solely of biologically active conjugates and/or a mixture of biologically inert compounds or carriers and biologically active conjugates.

Examples 7, 8, and 9 illustrate certain aspects of the invention with regard to the charged PEG linkers, and should not be construed as limiting the scope of the invention.

Although the invention herein has been described with reference to particular embodiments and examples, it is to be understood that these embodiments are merely illustrative of the principles and applications of the present invention. Other embodiments have been suggested and still others may occur to those skilled in the art upon a reading and understanding of the specification. It is intended that all such embodiments be included within the scope of this invention. It is therefore to be understood that numerous modifications may be made to the illustrative embodiments and that other arrangements may be devised without departing from the spirit and scope of the present invention as defined by the claims.

### EXAMPLES

The following examples describe the syntheses of the novel PEG linkers, including PEG linker compounds with mixed functional linkages, pyramidal PEG linkers, pyramidal multi-branched PEG linkers, and charged PEG linkers. The term mPEG denotes methoxy terminated polyethylene glycol.

### Example 1

### mPEG-urethane-succinimidyl ester (SU-PEG)

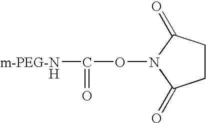

A solution of 5 KDa mPEG-NH2 (10 g, 2.0 mmol) and N,N′-disuccinimidyl carbonate (DSC, 4.1 g, 16.0 mmol) in a solvent mixture of methylene chloride (425 mL) and dimethylformamide (225 mL was added 4-dimethylaminopyridine (1.95 g, 16 mmol), and the reaction mixture was stirred at room temperature overnight. The reaction solution was then concentrated in vacuo, and the product was precipitated by the addition of ethyl ether, filtered, and crystallized from ethyl acetate to yield 8.5 g of mPEG-urethane-succinimidyl ester.

The PEG-urethane-succinimidyl ester (SU-PEG) is capable of reacting with an amino group to form a carbamide bond. The activated SU-PEG (PEG-urethane) linkers, e.g. SU-PEG succinimidyl ester, when compared to conventional active PEG ester, e.g. PEG SC-PEG (PEG-carbonate), are less reactive to hydrolysis in aqueous solution.

The PEG-urethane-p-nitrophenyl ester is also capable of reacting with an amino group on proteins. It can be synthesized by the reaction of PEG-NH2 with p-nitrophenyl chloroformate.

### Example 2

### mPEG-serine-carbamate-carbamide Mixed Linkages

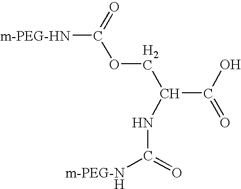

To a solution of L-serine methyl ester HCl (46.8 mg, 0.3 mmol) in 30 mL solvent mixture of anhydrous chloroform and DMF was added 5 KDa mPEG-urethane-succinimidyl ester (3 g, 0.6 mmol), dibutyltin dilaurate (300 mg, 0.48 mmol) and DMAP (73.4 mg, 0.6 mmol), and the reaction mixture was stirred at 60° C. for 18 hours. After partial removal of the solvent in vacuo, the product was precipitated with addition of ethyl ether, filtered, and crystallized from 2-propanol to give mPEG-serine-carbamate-carbamide methyl ester derivative. The mPEG-serine-carbamate-carbamide methyl ester was dissolved in water, and was then saponified by adjusting solution to pH 12 with 1 N NaOH, stirred for 2 hours at room temperature. The reaction mixture was then adjusted to pH 3 and extracted with methylene chloride. The combined organic extracts were concentrated in vacuo. And the product was precipitated with addition of ethyl ether, filtered, and crystallized from 2-propanol to give 2.1 g of mPEG-serine-carbamate-carbamide.

### Example 3

### Succinimidyl ester of mPEG-serine-carbamate-carbamide Mixed Linkages

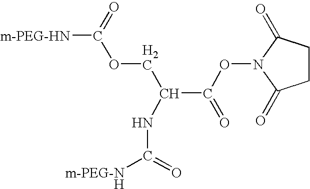

A solution of mPEG-serine-carbamate-carbamide (2 g, 0.2 mmol) and N-hydroxysuccinimide (23 mg, 0.2 mmol) in anhydrous methylene chloride was cooled to 0° C. in an ice bath and N,N′-dicyclohexylcarbodiimide (41.4 mg, 0.2 mmol) was added with stirring. The mixture was kept at 4° C. in refrigerator overnight. The precipitate was removed by filtration and the solution was concentrated in vacuo. The product was precipitated by addition of ethyl ether, filtered, crystallized from ethyl acetate to yield 1.8 g of succinimidyl ester of mPEG-serine-carbamate-carbamide mixed linkages.

### Example 4

### O-t-Butyldimethylsilyl-N-(2-hydroxyethyl) iminodiacetic acid

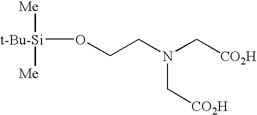

N-(2-Hydroxyethyl) iminodiacetic acid (HEIDA, 1 g, 5.6 mmol) was added to a solution of t-butyldimethylsilyl chloride (2.96 g, 19.6 mmol) in 6 mL of acetonitrile. After cooling to 4° C., 1,8-diazabicyclo[5.4.0]undec-7-ene (3.12 g, 20.6 mmol) was added and the reaction mixture was stirred for 18 hours at room temperature. The mixture was then extracted with n-hexane, and the combined organic extracts were evaporated to dryness in vacuo. The residue was dissolved in a solvent mixture consisting of MeOH (16 mL), tetrahydrofuran (8 mL), and water (8 mL), and then treated with 12 mL of 2N sodium hydroxide solution. The mixture was stirred for 2 hours at room temperature, and then was adjusted to pH 6 with 1N HCl. The solution was dried under vacuum, and the oily residue was triturated with ethyl ether. The precipitate obtained was purified from silica gel column chromatography in an eluting solvent mixture, methanol:ethyl acetate (1:1). The collected fractions were combined and dried to yield 0.64 g of O-t-butyldimethylsilyl-N-(2-hydroxyethyl) iminodiacetic acid.

### Example 5

### Pyramidal HEIDA-mPEG2

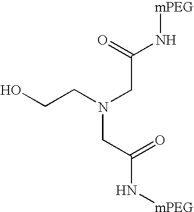

A solution of O-t-butyldimethylsilyl-N-(2-hydroxyethyl) iminodiacetic acid (0.118 g, 0.4 mmol), 5 KDa mPEG-NH2 (4 g, 0.8 mmol) and DMAP (97.6 mg, 0.8 mmol) in a solvent mixture of dry methylene chloride and DMF, 1-[3-(dimethylamine)propyl]-3-ethylcarbodiimide hydrochloride was added and stirred overnight. The solution was then concentrated in vacuo, and the product was precipitated with ethyl ether, and filtered to give O-t-butyl dimethylsilyl-HEIDA-mPEG2. The crude product was dissolved in a solution of acetic acid, water and THF stirred overnight. The reaction solution was then concentrated in vacuo and extracted with methylene chloride. The combined organic extracts were concentrated, precipitated by addition of ethyl ether, filtered, crystallized from 2-propanol to yield 2.7 g of HEIDA-mPEG2.

### Example 6

### Succinimidyl Carbonate of HEIDA-mPEG2

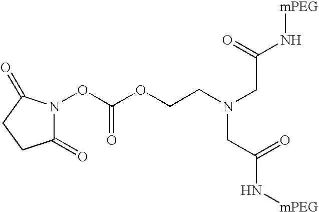

To a solution of 2 g of HEIDA-mPEG2 and DSC (0.41 g, 1.6 mmol) in a mixture of methylene chloride (40 mL) and DMF (22 mL) was added DMAP (0.2 g, 1.6 mmol) and the reaction mixture was stirred overnight. The reaction solution was then concentrated, and the product was precipitated by addition of ethyl ether, filtered, crystallized from ethyl acetate to give 1.7 g of Succinimidyl carbonate of HEIDA-mPEG.

### Example 7

### N-Fmoc-Lysine-(N-ε)-Diethylenetriaminepentaacetic acid

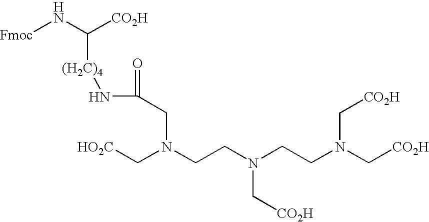

A solution of diethylenetriaminepentaacetic dianhydride (cDTPA, 5.14 g, 14.3 mmol) and N-hydroxylsuccinimide (3.32 g, 28.8 mmol) in 130 mL of dry DMF was stirred at 65° C. for 1 hour. The reaction mixture was cooled to room temperature and then mixed with a solution of N-Fmoc-(N-α)-lysineHCl (0.81 g, 2 mmol) and N,N-diisopropylethylamine (0.37 mL) in DMF (29 mL). The reaction mixture was stirred at room temperature for 3 hours and then was evaporated to dryness under vacuum. The oily residue was triturated with 1 mM HCl solution, and the solid was collected and purified by C18 chromatography to give 3.5 g N-Fmoc-Lysine-Diethylenetriaminepentaacetic acid.

### Example 8

### Lysine-(N-ε)-DTPA-mPEG5

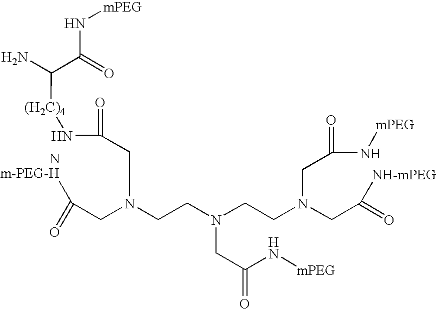

To a solution of N-Fmoc-lysine-(N-ε)-DTPA (125 mg, 0.16 mmol) and DIEA (103 mg, 0.8 mmol) in 5 mL of dry DMF was cooled to 0° C. in an ice bath was added isobutyl chloroformate (104 μL, 0.8 mmol). The reaction was stirred at 0° C. for 0.5 hour under nitrogen. A solution of 5 KDa mPEG-NH2 (4 g, 0.8 mmol) and DMAP (0.1 g, 0.8 mmol) in a mixture of anhydrous methylene chloride and DMF was cooled to 0° C. and added to the mixed anhydride reaction mixture. The reaction was stirred for 4 hours at 0° C. and then an additional 4 hours at room temperature. Piperidine (1 ml) was then added to the solution, and the reaction mixture was stirred at room temperature for 1 hour. The solution was concentrated under vacuum, and the product was precipitated by the addition of ethyl ether, filtered, and crystallized from 2-propanol to give Lysine-(N-ε)-DTPA-mPEG5.

Lysine-(N-ε)-DTPA-mPEG5 can be functionalized by the compounds capable of activating the amino groups, such as N,N′-Disuccinimidyl carbonate (DSC), N,N′-carbonyl diimidazole (CDI), or p-nitrophenyl chloroformate to yield the activated succinimidyl carbonate, imidazole carbamate or p-nitrophenyl carbonate, respectively. The activated forms of Lysine-(N-ε)-DTPA-mPEG5 can be coupled to the amino groups on proteins.

### V. Pro-Biomolecule PEG Conjugation

A drawback for Pegylation is the significant loss of biological activity of therapeutical proteins. For example, the pegylated interferons, such as Pegasys and Peg-Intron only retain 7 and 28% specific antiviral activity of the unmodified Interferon α-2a and Interferon α-2b, respectively. The numbers and sizes of PEG have significant impacts on protein drug's biological activity, pharmacokinetic and pharmacodynamic properties.

The present invention provides for attachment of novel PEG linker compounds with mixed functional linkages for Pegylation of biologically active molecules to produce Probiomolecule-PEG constructs. The Probiomolecule-PEG construct is the prodrug of biomolecule-PEG conjugate or biomolecule. The Probiomolecule-PEG conjugate will gradually lose portions (or all) of its PEG polymers in vivo to convert into smaller size biomolecule-PEG conjugate (or biologically active molecule), thereby increasing their biological activity in vivo. The Probiomolecule-PEG construct approaches can be applied to therapeutic biologically active molecules for providing long-acting and continuous-release biomolecule-PEG or biologically active molecule to result in a number of potential clinical advantages such as increased biological activity, sustained biological activity, sustained absorption, longer circulating half-life, protection against enzymatic degradation, reduced immunogenicity and toxicity, better physical and thermal stability, and enhanced potency. Furthermore, the Probiomolecule-PEG construct can increase and maintain high levels of bioactivity in vivo and meantime maximize biologically active molecule concentration in plasma for achieving better drug efficacy and safety.

For example, the sustained antiviral activity of Prointerferon-PEG construct in vivo will have better drug potency and clinically, may result in achieving greater sustained virological response and reducing the disease treatment period for hepatitis C patients.

For protein, the Proprotein-PEG construct is a novel protein drug delivery technology that provides controlled continuous-release system for delivering the more active pharmaceutical protein-PEG conjugate or protein.

The present invention for attachment of the mixed functional linkages of PEG compound to biologically active molecule provides two types of Probiomolecule-PEG conjugates, including the cleavable branched or linear PEG polymers. Each type of Probiomolecule-PEG conjugates can generate more active biomolecule-PEG or biologically active molecule in vivo depending on the functionality of the covalent bonds.

Type I is the Probiomolecule-PEG contains branched PEG linker compounds with permanent and releasable linkages. The Probiomolecule-PEG is gradually converted into biologically active molecule-PEG in vivo if the mixed functional linkages contain at least one permanent PEG linkage. The Probiomolecule-PEG can also be converted into biologically active molecule if all mixed functional linkages are releasable in vivo. The number of branched PEG polymers attached to biologically active molecules can vary from one single to multiple branched PEG polymers.

An example of Probiomolecule-PEG with two mixed functional linkages of PEG polymers is described as the following formula:

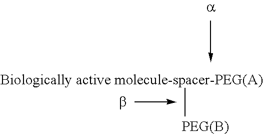

Formation of biologically active molecule-PEG, α bond is a permanent linkage and β bond is a releasable linkage in vivo.

Formation of biologically active molecule, both α and β bonds of the Probiomolecule (α, β)-PEG construct are releasable in vivo.

Sizes of PEG(A) and PEG(B) polymers can have equivalent or nonequivalent molecular weight.

For example, the Proprotein-PEG is constructed by connecting protein with mPEG-tyrosine(α, β)-carbamate-carbonate linker, and the mPEG-carbonate fragment is released in blood plasma resulting in a stable protein-mPEG-tyrosine-carbamate conjugate, which has higher activity in vivo.

Type II is the Probiomolecule-PEG attached with a linear but cleavable PEG linkages. The number of PEG polymers attached to biologically active molecules can varies from one single polymer strand to multiple polymer strands. The formula of Probiomolecule-PEG connected with a linear PEG strand containing two pieces of PEG polymers is shown as follows:

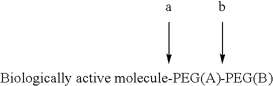

PEG(A) and PEG(B) can be either the same or different sizes of PEG polymers. A spacer may be implanted between biomolecule and PEG(A).

Formation of biologically active molecule-PEG conjugate, the covalent bond “b” between PEG(A) and PEG(B) is releasable in vivo. The releasable bond can be carboxylic ester, carbonate, sulfonic ester, phosphoric ester, carbamate-imidazo linkage, disulfide, or the pH or enzyme dependant releasing linkages. The covalent bond “a” between biologically active molecule and PEG(A) is a permanent linkage. The permanent bond can be carbamate, carbamide, amide, imide, sulfide, etc.

Formation of biomolecule or biomolecule-spacer, both a and b bonds are releasable in vivo. The spacer can be designed for a specific use. It can be a drug, or an enhancer for permeating the blood brain barrier, or an enhancer improving biomolecule's efficacy.

Biologically active molecules include, but not limited to, protein, peptide, oligopeptide, polypeptide, nucleic acid, DNA, RNA, oligonucleotide, oligosaccharide, polysaccharide, hormone, neurotransmitter, carbohydrate, sugar, disaccharide, lipid, phospholipid, glycolipid, sterol, amino acid, nucleotide, cell permeable peptide, small molecular drugs, etc. Potential biomoecules for the applications of PEG mixed functional linkages for Probiomolecule-PEG include, but not limited to, epoetin alfa, filgrastim, etanercept, interferon α-2a, interferon α-2b, interferon alfacon-1, interferon β-1a, interferon β-1b, interferon γ-1b, interleukins, insulin, urokinase, streptokinase, uricase, superoxide dismutase, asparaginase, arginine deaminase, glucocerebrosidase, galacosidase, retelapse, rasburicase, laronidase, oprelvekin, dornase α, collagenase, anistreplase, agalsidase, growth factors, hemoglobin, blood factors VII, VIIa, VIII, and IX, etc.

The present invention provides the use of Probiomolecule-PEG (Types I and II) for delivering neuropeptides to increase their bioavailability to the brain. The Probiomolecule-PEG technology is particularly useful for peptide hormones, growth factors, opioid peptides that have poor metabolic stability and low bioavailability. The Probiomolecule-PEG approach can also be applied to CNS drugs to enhance their bioavailability and blood brain barrier permeability.

The present invention provides the Promolecule-PEG technology to be used for the preparation of oral and nanoparticle drugs.

The present invention provides the new generation of PEG derivatives to be used for the preparation of oral and nanoparticle drugs. The new generation of PEG derivatives include PEG mixed functional linkages, pyramidal PEG polymers, multi-branched PEG polymers and charged PEG polymers.

The mixed functional linkages on the PEG compound are marked with the symbols: α, β, γ, δ, etc.

The PEG polymers attached to the different functional groups on spacers (tyrosine, serine, etc.) can have equivalent or different molecular weight.

### Example 9

### 40K mPEG-tyrosine (α, β) carbamate-carbonate Mixed Linkages

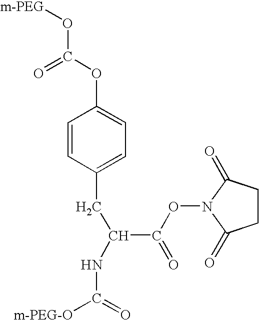

To a solution of 20K mPEG p-nitrophenyl carbonate (5 g, 0.25 mmol) in 50 mL of anhydrous methylene chloride was added tyrosine-O-t-butyl ester (29.5 mg, 0.125 mmol) and DMAP (46 mg, 0.38 mmol), and the reaction solution was refluxed overnight. After partial removal of the solvent under vacuum, the product was precipitated with addition of ethyl ether, filtered, and crystallized from ethyl acetate to give 40K mPEG-tyrosine-carbamate-carbonate t-butyl ester. The 40K mPEG-tyrosine-carbamate-carbonate t-butyl ester was then dissolved in a solvent mixture of 40% formic acid/methylene chloride and stirred for 3 hours at room temperature. The solution was concentrated in vacuo and precipitated with addition of ethyl ether, filtered, and crystallized from ethyl acetate to give 4.3 g of 40K mPEG-tyrosine-carbamate-carbonate.

### Example 10

### Succinimidyl ester of 40K mPEG-tyrosine (α, β) carbamate-carbonate Mixed Linkages

A solution of 40K mPEG-tyrosine-carbamate-carbonate (4 g, 0.1 mmol), N-hydroxysuccinimide (46 mg, 0.4 mmol) and DMAP (49 mg, 0.4 mmol) in anhydrous methylene chloride was cooled to 0° C. in an ice bath and 1-[3-(dimethylamino)propyl]-3-ethylcarbodiimide hydrochloride (EDC, 78 mg, 0.4 mmol) was added with stirring. The mixture was brought to room temperature after one hour and was stirred overnight. The solution was concentrated in vacuo. The product was precipitated with ethyl ether, filtered, and crystallized from ethyl acetate to give 3.4 g of succinimidyl ester of 40K mPEG-tyrosine-carbamate-carbonate.

Proprotein-PEG attached with mixed functional groups is designed to reduce PEG size in vivo and subsequently restore protein activity. For example, proteins connected with the mPEG-tyrosine (α, β)-carbamate-carbonate, which contains a permanent carbamate and a releasable aryl carbonate bonds linking to mPEG polymers. The aryl carbonate bond is cleavable in blood plasma but the carbamate bond is stable in plasma. After the release of PEG carbonate fragment in blood, the branched mPEG-tyrosine (α, β)-carbamate-carbonate is sequentially converted into a linear mPEG-tyrosine-carbamate linker.

### Example 11

### PEG Linker Compounds with Mixed Functional Linkages in Human Plasma

Experiments for the release of PEG fragment from PEG linker compounds containing releasable and permanent linkages were conducted in human plasma at 37° C. The PEG linker compounds used for the study were 40K mPEG tyrosine(α, β) carbamate-carbonate, 10K mPEG tyrosine (α, β) carbamate-carbonate, and 10K mPEG serine (α, β) carbamate-carbonate. Aliquot samples at 5 mg/mL in human plasma from each compound were incubated at 37° C. for various periods of time, ranging from 0.5 to 36 hours. Plasma samples at different time points were treated with the acetonitrile/methanol organic solvent mixture, vortexed, centrifuged, and dried in speed vacuum dryer. The residues were reconstituted in 50 mM phosphate buffer, pH 6, and the aqueous samples along with standard PEG markers were analyzed on Tris-Acetate 3-8% SDS-PAGE gel with iodine stain.

The plasma T_{1/2} for 40K mPEG tyrosine(α, β) carbamate-carbonate, 10K mPEG tyrosine (α, β) carbamate-carbonate, and 10K mPEG serine (α, β) carbamate-carbonate were 4.6, 1.9 and 4.8 hours, respectively.

### Example 12

### mPEG-urethane-benzaldehyde

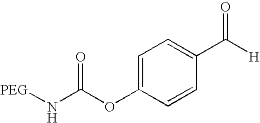

To a solution of 5 KmSU-PEG (1 g, 0.2 mmol) and 4-hydroxybenzaldehyde (196 mg, 1.6 mmol) in anhydrous chloroform (16 mL) was added triethylamine (162 mg, 1.6 mmol), and the reaction solution was stirred at 60° C. under nitrogen overnight. The solution was concentrated under reduced pressure, and the crude product was precipitated by addition of ethyl ether and filtered. The crude product was then dissolved in DMF followed by the addition of ethyl ether to precipitate the product. The solid product was filtered and washed with ethyl ether, and dried in vacuum to give 0.93 g of mPEG-urethane-benzaldehyde.

mPEG-urethane-benzaldehyde contains an aromatic aldehyde and a releasable urethane linkage. This PEG can be attached specifically to the protein N-terminal amino acid at lower pH, ranging from 4 to 6. Also, the protein-PEG-urethane-benzaldehyde conjugate may restore its activity in vivo via the release the unstable urethane linkage.

### Example 13

### PEGylation of Lysozyme with mPEG-urethane-benzaldehyde

Lysozyme (10 mg, 7×10⁻⁴ mmol) from chicken egg white was dissolved in 2 mL of 100 mM phosphate buffer, pH 5.3. To the protein solution was added 5 KmPEG-urethane-benzaldehyde (35 mg, 7×10⁻³ mmol) and sodium cyanoborohydride (2.5 mg, 0.04 mmol) and the reaction solution was stirred at room temperature overnight. The monopegylated Lysozyme conjugate was estimated to be over 50% by PAGE electrophoresis (NuPAGE 4-12% Bis-Tris gel), and the PEG is essentially attached to the N-terminal amino acid via a reductive amination reaction.

1. (canceled)

2. (canceled)

3. The linker compound of claim 13 wherein the mixed functional group linkages are selected from the group consisting of carboxylic esters, carbonates, carbamates, carbamides, amides, sulfides, disulfides, sulfonic esters and phosphoric esters.

4-12. (canceled)

13. A PEG linker compound comprising the formula:
Pn-Ln-R—C
wherein P is PEG moiety selected from the group consisting of PEG polymers, PEG derivatives, and PEG-lipids;
L is functional linkage moiety;
n is the number of different kinds of linkage moieties and n≧2;
R is a compound base structure bonded to at least two different kinds of functional groups for linking to P; and
C is a coupling group capable or reacting with a biologically active molecule.

14. The linker compound of claim 13, having the structure:

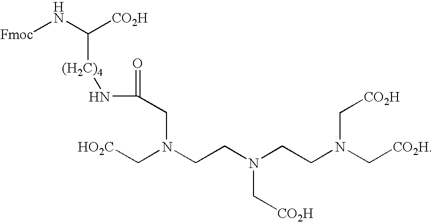

15. The linker compound of claim 13, having the structure:

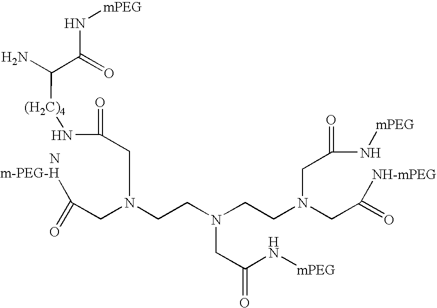

16. (canceled)

17. (canceled)

18. A compound formed by reacting the linker compound as set forth in claim 13 and a biologically active molecule.

19. A biologically active conjugate formed by reacting a biologically active molecule with the coupling group of the PEG linker compound as set forth in claim 13.

20. (canceled)

21. (canceled)

22. A method of preparing a biologically active conjugate comprising:
reacting a biologically active molecule with a PEG linker compound comprising the formula:
Pn-Ln-R—C
wherein P is PEG moiety selected from the group consisting of PEG polymers, PEG derivatives, and PEG-lipids;
L is functional linkage moiety;
n is the number of different kinds of linkage moieties and n≧2;
R is a compound base structure bonded to at least two different kinds of functional groups for linking to P; and
C is a coupling group capable or reacting with a biologically active molecule.

23-26. (canceled)

27. A nanoparticle formed from a PEG linker compound as set forth in claim 22.

28. A biologically active conjugate formed by reacting a biologically active molecule with the coupling group of the PEG linker compound as set forth in claim 22.

29. A biologically active conjugate as set forth in claim 28 wherein the biologically active molecule is selected from the group consisting of proteins, peptides, oligonucleotides, saccharides, lipids, liposomes and particulates, biomaterials, pharmaceuticals, vitamins, nucleic acids, amino acids, polypeptides, enzyme cofactors, steroids, carbohydrates, heparin, metal containing agents, receptor antagonists, receptor agonists, receptors or portions of receptors, extracellular matrix proteins, cell surface molecules, antigens, haptens, and small molecules.

30. A biologically active conjugate as set forth in claim 28 wherein the biologically active molecule is a protein selected from the group consisting of cytokines, enzymes, growth factors, monoclonal antibody, antibody fragments, single-chain antibody, albumin, immunoglobulins, clotting factors, somatropin, amylase, lipase, protease, cellulose, urokinase, galactosidase, staphylokinase, hyaluronidase, and tissue plasminogen activator.

31. A biologically active conjugate as set forth in claim 28 wherein the biologically active molecule is selected from the group consisting of epoetin alfa, filgrastim, etanercept, interferon α-2a, interferon α-2b, interferon alfacon-1, interferon β-1a, interferon β-1b, interferon γ-1b, interleukins, insulin, urokinase, streptokinase, uricase, superoxide dismutase, asparaginase, arginine deaminase, glucocerebrosidase, galacosidase, retelapse, rasburicase, laronidase, oprelvekin, dornase α, collagenase, anistreplase, agalsidase, growth factors, hemoglobin, and blood factors VII, VIII, and IX.

32. A Probiomolecule-PEG conjugate comprising two different function group linkages and a spacer group bonded to a biologically active molecule according to the following formula: 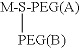
wherein M is a biologically active molecule;
S is a spacer group;
PEG(A) and PEG(B) are PEG polymers, PEG derivatives or PEG lipids; and
and at least one of the linkages is a releasable linkage in vivo.

33. A Probiomolecule-PEG conjugate as set forth in claim 32, wherein both of the linkages between S and PEG(A) and PEG(B) are releasable in vivo.

34. A Probiomolecule-PEG conjugate as set forth in claim 32, wherein PEG(A) and PEG(B) have equivalent or non-equivalent molecular weights.

35. (canceled)

36. A Probiomolecule-PEG conjugate as set forth in claim 32, wherein the molecular PEG(A) and/or PEG(B) is in the range of about 200 to about 150,000 Daltons.

37. (canceled)

38. A Probiomolecule-PEG conjugate as set forth in claim 32, wherein the releasable linkage is selected from the group consisting of carboxylic ester, carbonate, sulfonic ester, phosphoric ester, carbamate-imidazo linkage, disulfide, or a pH or enzyme dependant releasing linkage.

39-54. (canceled)

55. A Probiomolecule-PEG conjugate as set forth in claim 32, wherein the biologically active molecule is selected from the group consisting of proteins, peptides, oligonucleotides, saccharides, lipids, liposomes and particulates, biomaterials, pharmaceuticals, vitamins, nucleic acids, amino acids, polypeptides, enzyme cofactors, steroids, carbohydrates, heparin, metal containing agents, receptor antagonists, receptor agonists, receptors or portions of receptors, extracellular matrix proteins, cell surface molecules, antigens, haptens, and small molecules.

56. A Probiomolecule-PEG conjugate as set forth in claim 32, wherein the biologically active molecule is a protein selected from the group consisting of cytokines, enzymes, growth factors, monoclonal antibody, antibody fragments, single-chain antibody, albumin, immunoglobulins, clotting factors, somatropin, amylase, lipase, protease, cellulose, urokinase, galactosidase, staphylokinase, hyaluronidase, and tissue plasminogen activator.

57. A Probiomolecule-PEG conjugate as set forth in claim 32, wherein the biologically active molecule is selected from the group consisting of epoetin alfa, filgrastim, etanercept, interferon α-2a, interferon α-2b, interferon alfacon-1, interferon β-1a, interferon β-1b, interferon γ-1b, interleukins, insulin, urokinase, streptokinase, uricase, superoxide dismutase, asparaginase, arginine deaminase, glucocerebrosidase, galacosidase, retelapse, rasburicase, laronidase, oprelvekin, dornase α, collagenase, anistreplase, agalsidase, growth factors, hemoglobin, and blood factors VII, VIII, and IX.
